(19) United States
(12) Patent Application Publication
(10) Pub. No.: US 2004/0035423 A1
(43) Pub. Date: Feb. 26, 2004

(54) System for physiological monitoring during sleep

(76) Inventors: Harry Louis Platt, Randwick (AU); Allan Michael Shell, Randwick (AU); Michael Anthony Romm, Randwick (AU); Vladimir Jankov, Randwick (AU)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(21) Appl. No.: 10/373,620

(22) Filed: Feb. 25, 2003

Related U.S. Application Data
(63) Continuation of application No. PCT/AU01/01055, filed on Aug. 24, 2001.

(30) Foreign Application Priority Data
Aug. 25, 2000 (AU) PQ9666

(51) Int. Cl. A61M 16/00

(52) U.S. Cl. 128/204.23; 128/204.22

(57) ABSTRACT

A diagnostic medical device which monitors physiological parameters during sleep is disclosed. The device includes mask means (2) adapted to fit over the nose and mouth of a patient being monitored, and an acquisition unit (5) is adapted to be removeably fitted to the mask means (2). The mask means (2) has inlet means (3) to allow the patient to breath, sensor means (8) which monitor physiological conditions and which provide physiological signals thereto for storage or real time data transmission to external devices.

The present invention relates generally to diagnostic medical devices and, in particular, to a diagnostic medical device which monitors physiological parameters during sleep.

### BACKGROUND TO THE INVENTION

Portable diagnostic medical devices are used for monitoring of biological signals of patients in order to detect disease. These devices are used both in the hospital environment and ambulatory settings.

Modern medical diagnostic devices are intelligent data loggers capable of acquiring, analysing and storing biological data into memory. Biological data can be acquired from a single sensor or from a multiplicity of sensors connected to the patient.

Usually sensors such as ECG electrodes, pulse oximetry emitter-detector couple, plethysmography electrodes, EEG electrodes, leg and chest movement sensors, body position sensors, etc are mounted on a patient's body by means of adhesive media or spring loaded clips. The sensors then connect to the monitor (logger) by means of leads and cables. The list of sensors above describes a typical setup for sleep disorders investigations; up to 20 different sensors, leads and cables can be attached to the data logger device.

There are several disadvantages in using these types of recording systems including: connecting wires, can be unintentionally pulled by the patient during the night can dislodge sensor(s), which will lead to the loss of data; setup and connection of multiple leads and cables requires an assistance of trained technician; long wires become a source of common mode noise in the sensitive front-end circuits of acquisition system, and; the high cost of this technology due to the number of sensors required.

It would be advantageous to locate biological sensors and data logging device in such a way that the number and length of leads and cables required is minimal.

### OBJECT OF THE INVENTION

It is an object of the present invention to provide a diagnostic medical device which monitors physiological parameters during sleep and method of its use which is simple to use, reliable and noise free for prolonged acquisition of multiple biological signals during sleep. At the very least, the object of the invention is to provide an alternative to known monitoring devices.

### DISCLOSURE OF THE INVENTION

According to one aspect of the present invention there is disclosed a diagnostic medical device which monitors physiological parameters during sleep, said device including-a mask means adapted to fit over the nose and mouth of a patient being monitored, an acquisition unit being adapted to be removeably fitted to the mask means, said mask means having inlet means to allow the patient to breath, wherein said acquisition unit including sensor means which monitor physiological conditions and which provide physiological signals thereto for storage or real time data transmission to external devices.

Preferably, the mask means is a standard nebuliser mask used for oxygen ventilation and respiratory support having ventilation holes for an external air supply.

Preferably, the acquisition unit is attached to the mask by means of anchors protruding through pre-punched holes in the soft plastic of the mask.

Preferably, the inlet means is adapted to be provided for positive pressure oxygen intake, connection of air mass flow measurement devices or air pressure sensors.

Preferably the sensor means are located internally or externally of the body of the acquisition unit. For instance, the acquisition unit has an air inlet which takes airflow from the mask for air flow and air pressure monitoring. Preferably, the outer diameter of the air inlet matches the standard size of an oxygen hose.

A temperature sensor is preferably positioned within the mask and an ear mounted pulse oximeter spring clip assembly is preferably connected to the acquisition unit via short leads. EEG electrodes, ECG and plethysmography leads are also connected to the acquisition unit via short leads. Preferably, a connector is used for recorded data uploading and internal battery re-charging or for a real time data transmission.

The system preferably monitors the following physiological signals:

air flow

air pressure

air temperature

ECG signal

EEG signal

plethysmography signal for chest movement monitoring oximetry.

### BRIEF DESCRIPTION OF THE DRAWINGS

The present invention will be now be described with reference to the accompanying drawings in which:

FIG. 1 is alschematic side view of a patient wearing a standard disposable nebuliser type mask;

FIG. 2 is a schematic side and front view of the patient and mask of FIG. 1 with an acquisition unit attached to the mask;

FIG. 3 is a schematic side and front view of the acquisition unit shown in FIG. 2 with sensor attachments thereto;

FIG. 4 is a schematic front view of the patient wearing the mask having the sensor attachments being attached between the acquisition unit and the relevant parts of the patient's body;

FIG. 5 is a bottom view of the nebuliser mask of FIG. 1; and

FIG. 6 is detailed view of the anchoring of the acquisition unit to the nebuliser mask.

### BEST MODE OF CARRYING OUT THE INVENTION

In the preferred embodiment a system for sleep investigation is described. The system preferably monitors the following physiological signals:

air flow

air pressure

air temperature

ECG signal

EEG signal

plethysmography signal for chest movement monitoring oximetry.

A standard disposable nebuliser type mask is used in the preferred embodiment of the invention, however, other types of masks are also within the scope of the invention. The standard masks are used for life supporting oxygen ventilation and respiratory support. The mask of the preferred embodiment as seen in FIG. 1 where a person 1 wears a disposable nebuliser type mask 2 having ventilation holes 3 on either side of the mask 2, the holes 3 providing for external fresh air. A standard size inlet 4 is used for positive pressure oxygen intake, connection of air mass flow measurement devices or air pressure sensors as will be described.

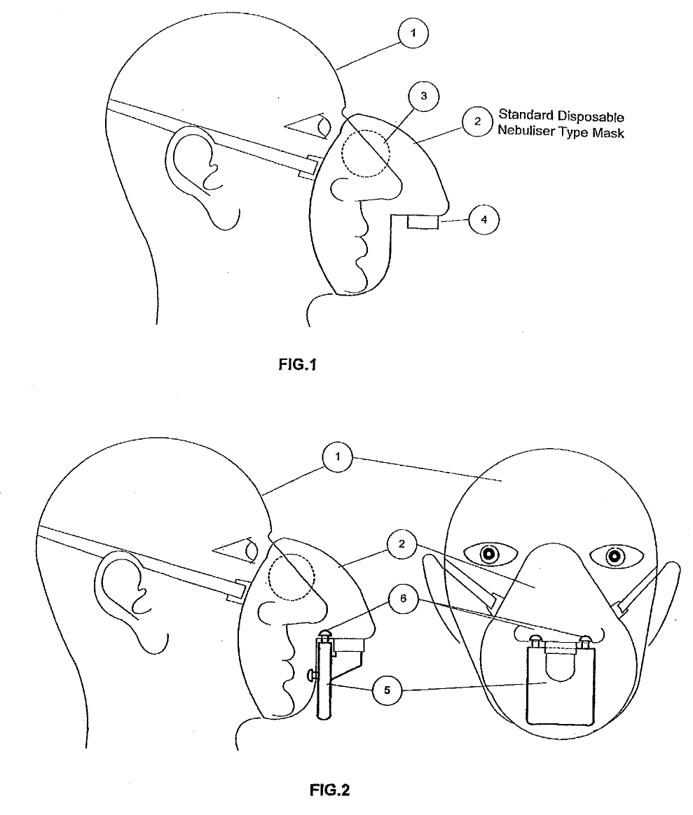

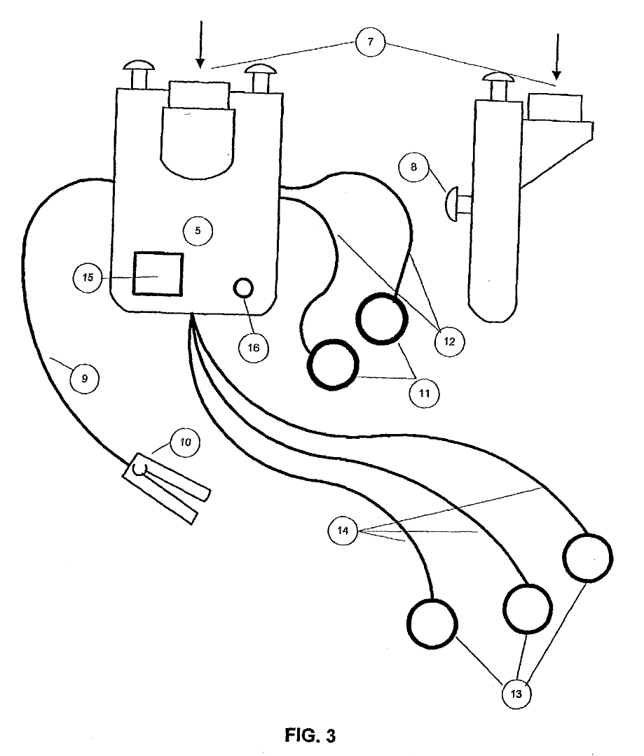

As seen in FIG. 2, an acquisition unit 5 is preferably attached to the mask 2 by means of two anchors 6, protruding through pre-punched holes in the soft plastic of the mask 2. This anchoring effectively locks into position the acquisition unit 5.

Referring to FIG. 3 where two views of the acquisition unit 5 are shown, an air inlet 7 of the acquisition unit 5 takes airflow from the mask 2 for air flow and air pressure monitoring. The outer diameter of inlet 7 matches the standard size of an oxygen hose (not illustrated). A temperature sensor 8 which is preferably positioned within the mask 2 partially protrudes into the mask via a pre-punched hole in the front surface of the mask 2. An ear mounted pulse oximeter spring clip assembly 10 is connected to the acquisition unit 5 via a short cable 9. EEG electrodes 11 are also connected to the acquisition unit 5 via short leads 12. Furthermore, ECG and plethysmography leads 13 are connected to the acquisition unit 5 via short leads 14 and a connector 15 is used for recorded data uploading and internal battery re-charging or for a real time data transmission. A LED 16 indicates progress of any data logging.

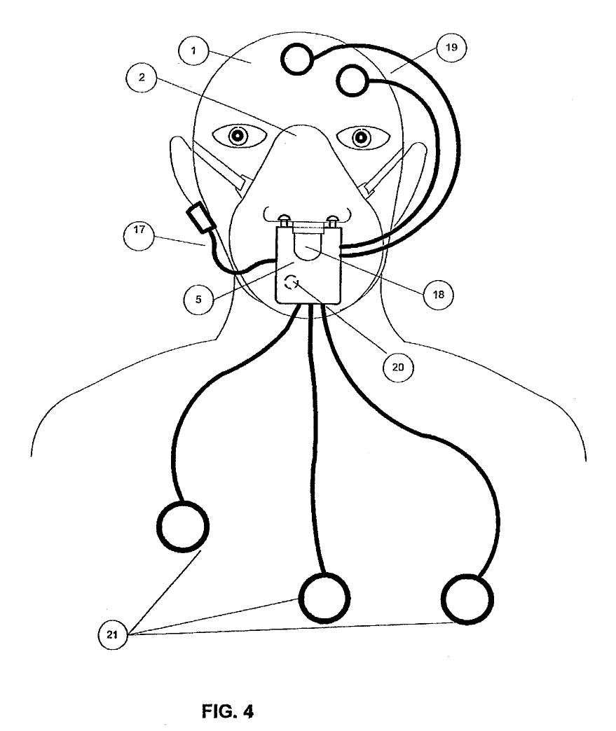

In use as seen in FIG. 4, the person 1 wears mask 2 with an acquisition unit 5 attached. Pulse oximetry is monitored via the oximeter 17, air flow and air pressure are monitored via the air inlet 18 of the mask 2, EEG signals are acquired via the EEG leads 19, air temperature is monitored via a thermistor 20 and ECG/plethysmography signals are monitored via leads 21.

The preferred embodiment of the invention as seen from FIG. 4, shows the cables attached to the head sensors are short and arranged for minimal impact from patient's movements in sleep. Leads for ECG/plethysmography signals are much shorter than leads used in standard sleep studies. Clearly since all the elements of the system are attached to the body, arrangement of sensors and setup of the system can be performed by the person with the assistance of a mirror.

The acquisition system and the body of the person form a closed, self-contained structure less susceptible to common mode electrical noise and sensor displacement due to any movements. If real time acquisition is conducted, only a single external connection using a light cable is required.

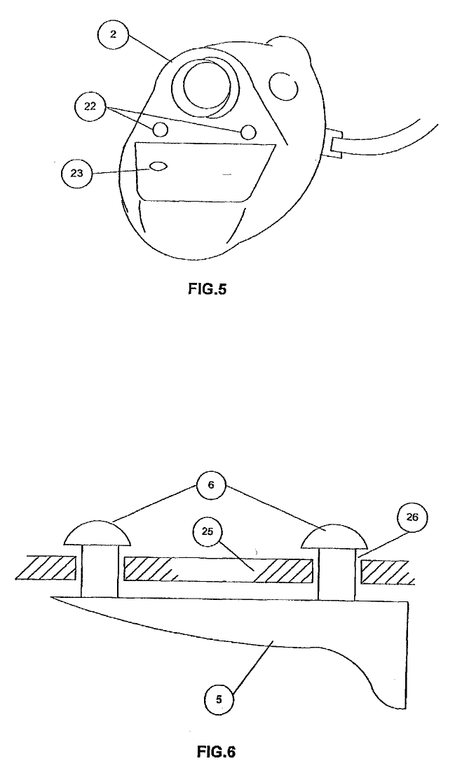

The disposable nebuliser type mask 2 with two pre-punched anchoring holes 22 and thermistor hole 23 is shown in FIG. 5. The position of the punched holes is defined by the shape of acquisition unit 5.

Referring to FIG. 6, a detailed view of the anchoring is shown. The position of acquisition unit 5 is located by the anchors 6 which protrude through the prepunched anchoring holes 26 in the soft plastic of the mask 25. The shape of the anchors 6 allows for a low-force attachment to the mask. By pulling the acquisition unit 5 with sufficient force, it can be separated from the mask for replacement and data transfer.

The foregoing describes only one embodiment of the present invention, and modifications obvious to those skilled in the art can be made thereto without departing from the scope of the present invention.

1. A diagnostic medical device which monitors physiological parameters during sleep, said device including mask means adapted to fit over the nose and mouth of a patient being monitored, and an acquisition unit being adapted to be removeably fitted to the mask means, said mask means having inlet means to allow the patient to breath, and wherein said acquisition unit includes sensor means which monitor physiological conditions and which provide physiological signals thereto for storage or real time data transmission to external devices.

2. A diagnostic medical device according to claim 1, wherein the mask means is a standard nebuliser mask used for oxygen ventilation and respiratory support having ventilation holes for an external air supply.

3. A diagnostic medical device according to either claim 1 or claim 2, wherein the mask is made from soft plastic and the acquisition unit is attached to the mask by means of anchors protruding through pre-punched holes in the soft plastic of the mask.

4. A diagnostic medical device according to any one of the preceding claims, wherein the inlet means is adapted to be provided for positive pressure oxygen intake, connection of air mass flow measurement devices or air pressure sensors.

5. A diagnostic medical device according to any one of the preceding claims, wherein the sensor means are located internally of the body of the acquisition unit.

6. A diagnostic medical device according to any one of claims 1 to 5, wherein the sensor means are located externally of the body of the acquisition unit.

7. A diagnostic medical device according to any one of the preceding claims, wherein a temperature sensor is positioned within the mask and an ear mounted pulse oximeter spring clip assembly is connected to the acquisition unit via short leads, and wherein EEG electrodes and ECG and plethysmography leads are also connected to the acquisition unit via short leads.

8. A diagnostic medical device according to any one of the preceding claims, wherein a connector is used for recorded data uploading and internal battery re-charging or for a real time data transmission.

9. A diagnostic medical device according to any one of the preceding claims, wherein the device monitors any one or more of the following physiological signals: air flow, air pressure, air temperature, ECG signal, EEG signal, plethysmography signal for chest movement monitoring, or oximetry.
